(19) United States
(12) Patent Application Publication
(10) Pub. No.: US 2009/0319044 A1
(43) Pub. Date: Dec. 24, 2009

(54) Methods and Compositions for Improving the Incorporation of Orthopaedic and Orthodontic Implants

(76) Inventor: Francis Young-In Lee, Fort Lee, NJ (US)

Correspondence Address: Bryan Cave LLP, 1290 Avenue of the Americas, 33rd Floor, New York, NY, 10104-3300, US

(21) Appl. No.: 12/223,503

(22) PCT Filed: Jan. 31, 2007
(86) PCT No.: PCT/US07/02564

§ 371 Date: Mar. 30, 2009

Related U.S. Application Data
(60) Provisional application No. 60/764,233, filed on Jan. 31, 2006.

(51) Int. Cl. A61F 2/28; A61L 27/54; A61P 19/00; A61B 17/58

(52) U.S. Cl. 623/16.11; 424/423; 606/86 R

(57) ABSTRACT

The present invention provides methods of improving the incorporation of an implantable device into a bone of a host in need thereof. More particularly, the methods of the present invention include implanting the device into the bone of a host, wherein the device is at least partially made of a non-metallic material. Disposed on at least one surface of the device is an amount of hydroxyapatite and bisphosphonate, which in combination, are effective to reduce osteolysis and improve incorporation of the implant into the host bone compared to an implant without the hydroxyapatite and/or bisphosphonate. The present invention also provides methods for making such implants, as well as, the implants themselves.

### CROSS-REFERENCE TO RELATED APPLICATIONS

This application claims priority to, and incorporates by reference, U.S. provisional patent application Ser. No. 60/764,233, filed Jan. 31, 2006.

### FIELD OF THE INVENTION

The present invention relates to orthopaedic and orthodontic implants, methods and compositions for improving the incorporation of such implants in a host, and methods of making such implants.

### BACKGROUND OF THE INVENTION

Orthopaedic implants are a mainstay of joint reconstruction surgeries. These surgeries are known to provide excellent pain relief and long-term functional improvement to a joint. Similarly, orthodontic and facial implants are routinely used during oral and/or cosmetic surgeries, which may be performed for reconstructive or cosmetic purposes. In many cases, however, certain polyethylene components of such implants generate particle wear debris in the periprosthetic space. The wear debris reaction that results often leads to periprosthetic bone loss (i.e., osteolysis) and aseptic loosening of the implant—a significant clinical problem.

Some implant designs have addressed this issue by providing a hydroxyapatite (HA) coating to such implants prior to insertion into a host. Such hydroxyapatite coatings have been shown to provide moderate improvements to the incorporation of such implants at the implant/host-bone interface. Despite such improvements, there is a continuing need for additional improved methods and compositions that further increase the incorporation of such orthopaedic and orthodontic implants in a host.

### SUMMARY OF THE INVENTION

One embodiment of the present invention is a method of improving the incorporation of an implantable device into a bone of a host in need thereof. This method includes implanting the device into the bone of a host, wherein the device is at least partially made of a non-metallic material. Disposed on at least one surface of the device is an amount of hydroxyapatite and bisphosphonate, which in combination, are effective to reduce osteolysis and improve incorporation of the implant into the host bone compared to an implant without the hydroxyapatite and/or bisphosphonate.

Another embodiment of the present invention is a method of making a device for implanting into a bone of a host in need thereof. This method includes contacting, prior to implantation, the device or at least one surface thereof with an amount of bisphosphonate and hydroxyapatite, which in combination, are effective to reduce osteolysis adjacent to the implant site and to improve incorporation of the implant into the host bone compared to a device without the hydroxyapatite and/or bisphosphonate.

Another embodiment of the present invention is an orthopaedic or orthodontic implant. This implant comprises a surface or component that is at least partially made of a non-metallic material. A surface or component of the device is provided with an amount of hydroxyapatite and bisphosphonate that is effective to reduce osteolysis adjacent to a site where the implant is implanted and to improve incorporation of the implant into a host bone compared to an implant without the hydroxyapatite and/or bisphosphonate.

### BRIEF DESCRIPTION OF THE FIGURES

FIG. 1 is a bar graph showing the selective induction of cell death in osteoclast precursors and osteoblasts.

FIG. 2 is a faxitron image of the femurs in the test and control rodents described in Example 1. The arrow identifies areas in which osteolysis was present in the femur of the control rodent.

FIG. 3 is a micro-CT image of the femurs in the test and control rodents described in Example 1. The arrow identifies areas in which osteolysis was present in the femur of the control rodent.

FIG. 4 is a bar graph comparing the bone mineral density of femurs taken from the control and test (i.e., zoledronate-treated) rodents, as described in Example 1. The left femur (titanium pin group) and right femur (plastic pin group) are also compared. “BMD” refers to the bone mineral density of the subject bone tissue.

FIG. 5 is a bar graph summarizing the results of the pull-out testing of femurs taken from the control and test (i.e., zoledronate-treated) rodents, as described in Example 1.

FIG. 6 is a bar graph, and related image, showing that hydroxyapatite-zoledronate implants exhibit higher peri-implant bone density in the presence of wear particle-induced inflammatory bone loss.

FIG. 7 is a bar graph summarizing the micro-CT measurements of the periprosthetic bone area of the femurs in the test and control rodents described in Example 3.

FIG. 8 is a bar graph summarizing the pull-out testing of the femurs in the test and control rodents described in Example 3.

FIG. 9 is a load-displacement curve, which compares the pull-out testing results that were obtained for the femurs in the test and control rodents described in Example 3.

FIG. 10 is a bar graph summarizing the pull-out energy testing of the femurs in the test and control rodents described in Example 3.

FIG. 11 is an x-y scatter plot showing the correlation of the pull-out force and bone area results described in Example 3.

FIG. 12 shows several bar graphs summarizing the UHMWPE wear particle analysis described in Example 4.

FIG. 13 shows several photographs of MC3T3-E1 cells engulfed in a clinically relevant amount of UHMWPE particles, as described in Example 4.

FIG. 14 (A) is a table summarizing the NFATc1 nuclear shuttling results described in the Example 4; (B) is a photograph showing nuclear translocation of NFATc1-GFP; (C) is a bar graph summarizing MC3T3-E1 calcineurin activity; and (D) is a bar graph summarizing MC3T3-E1 NFATc1 Activity.

FIG. 15 (A) is a set of photographs showing the nuclear translocation of NFATc1-GFP in RAW 264.7 cells; and (B) is a bar graph summarizing the data described in Example 4 relating to the activation of calcineurin by UHMWPE wear particles.

FIG. 16 is a set of bar graphs showing the expression levels of COX-2 and TNF-alpha following A23187 treatment in MC3T3 cells.

FIG. 17 is a set of bar graphs showing (i) NFATc inhibition and RANKL promoter activity and (ii) RANKL to OPG ratios as measured by ELISA, as described in Example 4.

FIG. 18 is a set of bar graphs showing (i) TNF-alpha gene induction by UHMWPE and (ii) the results of the TNF-alpha promoter-luciferase assay in RAW 264.7 cells described in Example 4.

FIG. 19 is a bar graph showing the M-CSF gene expression data that are described in Example 4.

### DETAILED DESCRIPTION OF THE INVENTION

In one embodiment of the present invention, a method of improving the incorporation of an implantable device into the bone of a host in need thereof is provided. In this embodiment, disposed on at least one surface of the device is an amount of hydroxyapatite and bisphosphonate, which in combination, are effective to reduce osteolysis and improve incorporation of the implant into the host bone compared to an implant without the hydroxyapatite and/or bisphosphonate. In this embodiment, the device is at least partially made of a non-metallic material.

As used herein, “incorporation” of the implantable device refers to the ability of such a device to be inserted into and reside within a bone (or bones) of a host, such that the device is not easily dislodged from its intended and/or desired location. In other words, for example, a device according to the present invention that is implanted into a bone of a host requires more force or work to dislodge it from the implant site compared to a similar device that does not have hydroxyapatite and bisphosphonate on its surface (or that has one, but not both, of the agents on its surface).

The devices of the present invention are effective to reduce osteolysis adjacent to the implant area caused, e.g., by particle wear debris. Furthermore, the devices of the present invention provide improved incorporation of the implant into the host's bone (or bones). The reduction in osteolysis and improved incorporation are a result of the presence on at least a portion of the implant's surface of hydroxyapatite and bisphosphonate.

As used herein, “an effective amount” of hydroxyapatite and bisphosphonate is that amount of each agent required to achieve reduced osteolysis and improved incorporation of the implant into the hosts bone (or bone) compared to an implant that is not coated with both hydroxyapatite and bisphosphonate (or is coated with only one of these agents). In the present invention, reduced osteolysis is confirmed using various imaging techniques, such as, for example, faxitron imaging or micro-CT imaging or DEXA scanning to measure bone mineral density adjacent to the implant site. (See, e.g., Example 1).

In the present invention, improved incorporation of the implant into the bone (or bones) of a host is confirmed using a pull-out test as described in Example 1. The devices according to the present invention preferably are expected to have a statistically significantly higher pullout energy compared to similar devices that are not coated with both hydroxyapatite and bisphosphonate (or are coated with only one of these agents).

As used herein, the term “host” means any mammal that may benefit from an implantable device according to the present invention. Preferably, the host is a human in need of such an implantable device.

The implantable devices of the present invention are preferably orthopaedic or orthodontic implantable devices for use in human and veterinarian applications. The implantable device of the present invention may be composed of any medically-suitable material. Non-limiting examples of such materials include inert metals, inert polymeric materials, ceramics, solid hydroxyapatite, and composite materials, such as metal-polymer combinations.

In a preferred embodiment, the implantable device or a component thereof is made of a polymeric material such as, for example, polylactic acid, polyglycolic acid, polydioxanone, tyrosine polycarbonate, polyethylene, and composites thereof. More preferably, the polymeric material is a medical grade polyethylene. In general, polyethylene is a porous synthetic polymer that is biologically inert and non-biodegradable in the body of a mammal. Many orthopaedic and orthodontic implants are made of polyethylene (and/or otherwise include polyethylene components), which are commonly used in, for example, orthodontic procedures and cosmetic surgery, e.g., in chin, cheek, and jaw line reconstruction. The porosity of polyethylene allows for soft tissue and vascular ingrowth, which preferably facilitates incorporation of the implant. In addition, polyethylene materials may be carved or contoured to fit within a particular three-dimensional space.

The implantable device also may be made of solid or substantially solid hydroxyapatite. Those of ordinary skill in the art will appreciate that solid (or substantially solid) pieces of hydroxyapatite material may also be carved and trimmed into a desired three-dimensional shape. Such hydroxyapatite implants have also been used, for example, in cosmetic surgery for cheek, chin, jaw, nose, and browbone facial reconstruction, treatment, and augmentation.

In the present invention, “implant,” “implantable device,” and “device” are used interchangeably and mean any medical device that currently exists or that is discovered hereafter, which is inserted into and/or attached, applied, or otherwise incorporated into a bone, bone material, cartilage/bone interface, tooth, or other similar biomaterial within a mammal. Such devices may be used for a variety of medical applications, including, for example, for joint replacement and repair, oral surgery, and cosmetic surgery (e.g., for cosmetic or reconstructive purposes, such as cheek, chin, jaw, nose, and browbone facial reconstruction). Non-limiting examples of implants for use in the present invention include spinal column implants, bone plates, external fixators, hip prosthesis, knee prosthesis, pins, screws, washers, nails, staples, bolts, mechanical fasteners, and the like.

The hydroxyapatite and bisphosphonate may be applied to a surface of the device or a component thereof using any of numerous methods known to those of ordinary skill in the art. For example, the hydroxyapatite and bisphosphonate compositions may be topically applied to a surface of the device, such as by immersion (dipping), coating, spraying, or by any other suitable means.

In the present invention, any medically appropriate form of bisphosphonate (also known as diphosphonate) may be used. For example, certain medically appropriate forms of bisphosphonate are commercially-available, have been approved for use by the appropriate regulatory agencies, have known toxicity profiles, and/or have known effective dose ranges. Many bisphosphonates have been used to prevent and/or treat osteoporosis, osteitis deformans, bone metastasis, multiple myeloma, and other bone-related diseases. In general, the family of currently-available bisphosphonate compositions consists of two groups, namely, Nitrogen-containing (“N-containing”) and non-Nitrogen-containing (“non-N-containing”) bisphosphonate compositions. The bisphosphonate family of compositions share the following chemical scaffold:

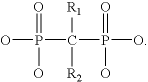

The “short side chain”, designated as R₁ above, is known to primarily affect the pharmacokinetics of a bisphosphonate composition. The “long side chain”, designated as R₂ above, is known to affect the chemical properties, mode of action, and relative strength or potency of a bisphosphonate composition. Non-limiting examples of medically appropriate bisphosphonate compositions that may be used in the present invention are summarized in the tables below:

N-Containing Bisphosphonate Compositions

| Composition | R₁ side chain | R₂ side chain | Supplier |
|---|---|---|---|
| pamidronate | —OH | —CH₂—CH₂—NH₂ | Novartis Pharmaceuticals |
| | | | (East Hanover, NJ) |
| neridronate | —OH | —(CH₂)₅—NH₂ | Abiogen Pharma SPA |
| | | | (Italy) |
| olpadronate | —OH | —(CH₂)₂N(CH₃)₂ | Gador Pharmaceuticals Labs |
| | | | (Argentina) |
| alendronate | —OH | —(CH₂)₃—NH₂ | Merck & Company, Inc. |
| | | | (Whitehouse Station, NJ) |
| ibandronate | —OH | 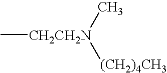 | Roche Therapeutics, Inc. (Nutley, NJ) |
| risedronate | —OH | 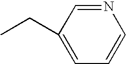 | Procter & Gamble Pharmaceuticals, Inc. (Cincinnati, OH) |
| zoidronate | —OH | 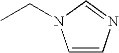 | Novartis Pharmaceuticals (East Hanover, NJ) |

Non-N-Containin Bisphosphonate Compositions

| Composition | R₁ side chain | R₂ side chain | Supplier |
|---|---|---|---|
| etidronate | —OH | —CH₃ | Procter & Gamble Company |
| | | | (Cincinnati, OH) |
| clodronate | —Cl | —Cl | Procter & Gamble Company |
| | | | (Cincinnati, OH) |
| tiludronate | —H | 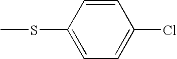 | Schering Oy (Finland) |

The amount of a bisphosphonate applied to an implant according to the present invention may vary depending on the type of bisphosphonate used. In addition, the method used to apply the bisphosphonate composition to the implant may affect the preferred concentration (or effective amount) of such bisphosphonate compositions. For purposes of illustration only, the preferred concentration (or effective amount) of bisphosphonate applied to an implant (or component thereof) may range from about 12 mM to about 80 mM or, preferably, from about 21 mM to about 65 mM or, still more preferably, from about 30 mM to about 50 mM. The bisphosphonate may be applied to the device alone or in combination with other medically appropriate buffers, salts, carriers, actives, and the like.

Any medically appropriate hydroxyapatite may be used in the present invention. In general, “hydroxyapatite” is a term used to describe a composition that is substantially similar to the mineral component of bones and teeth in mammals. Hydroxyapatite is generally represented by the formula: Ca₁₀(PO₄)₆(OH)₂. As its formula suggests, hydroxyapatite consists of Ca²⁺ ions surrounded by PO₄³⁻ and OH⁻ ions. Such hydroxyapatite compositions are known to be susceptible to substitution. In particular, the hydroxyls are known to be especially susceptible to substitution. For example, it has been shown that one or more of the hydroxyls may be substituted with a carbonate group, fluoride ion, and/or chloride ion. Accordingly, the invention provides that the hydroxyapatite compositions used in the present invention may represent various analogs, derivatives, variations, or combinations thereof. Such hydroxyapatite compositions are commercially-available from any of numerous vendors, such as Berkeley Advanced Biomaterials, Inc. (Berkeley, Calif.) and Howmedica, Inc. (Rutherford, N.J.).

The amount of hydroxyapatite used in the present invention may vary depending on the type of hydroxyapatite used and/or the method employed to provide the hydroxyapatite to the implant. Methods of applying hydroxyapatite to an implant are well-known and include for example, spraying, dipping, electrophoresis, and the like. Typically, the hydroxyapatite forms a layer on a surface of the implant that is approximately 1-100 μm thick, preferably, 1-50 μm, more preferably 1-40 μm, including 1-30 μm, 1-20 μm, 1-10 μm, 1 μm, 2 μm, 3 μm, 4 μm, 5 μm, 6 μm, 7 μm, 8 μm, 9 μm, and 10 μm thick. In addition, multiple layers of hydroxyapatite may be applied to the implant. Hydroxyapatite coated implants are also commercially available from a variety of manufacturers, such as, for example Stryker (Kalamazoo, Mich.)

Another embodiment of the invention is a method of making a device for implanting into a bone of a host in need thereof. This method includes contacting, prior to implantation, the device or at least one surface thereof with an amount of bisphosphonate and hydroxyapatite, which, in combination, are effective to reduce osteolysis adjacent to the implant site and to improve incorporation of the implant into the host bone compared to a device without the hydroxyapatite and/or bisphosphonate.

In this method, the contacting step may be accomplished by immersion (dipping), coating, spraying, or any other suitable means so that at least one surface of the implant that will come in contact with the host's bone has disposed thereon hydroxyapatite and bisphosphonate.

The hydroxyapatite and bisphosphonate may be applied to a surface of the device together as a single composition. Alternatively, each agent may be applied separately. For example, the device may be dipped into a composition that includes hydroxyapatite. The device may then be allowed to dry. Thereafter, the device containing a coating of hydroxyapatite may then be immersed in a composition that includes bisphosphonate. Thereafter the device is allowed to dry and is subsequently implanted into a host.

The bisphosphonate and hydroxyapatite preferably are applied to the device as over-lapping layers with the hydroxyapatite layer applied to the device first. In the present invention, the entire device may be coated with bisphosphonate and hydroxyapatite. Alternatively, only select portions of the device may be so coated (e.g., the ends of the device).

The hydroxyapatite and bisphosphonate compositions may, alternatively, be applied to internal portions of the implant, such that the compositions are gradually released overtime. In such embodiments, the implant may comprise pores or be made of a material that enables the hydroxyapatite and bisphosphonate compositions to be gradually released over time.

In this method, the bisphosphonate and hydroxyapatite are as described above.

Another embodiment of the invention is an orthopaedic or orthodontic implant. This implant comprises a surface or component that is at least partially made of a non-metallic material. A surface or component of the device is provided with an amount of hydroxyapatite and bisphosphonate that is effective to reduce osteolysis adjacent to a site where the implant is implanted and to improve incorporation of the implant into a host bone compared to an implant without the hydroxyapatite and/or bisphosphonate.

In this embodiment, the implant, bisphosphonate, and hydroxyapatite are all as described above.

According to additional embodiments of the present invention, the orthopaedic or orthodontic implantable devices may further be provided with one or more NFAT inhibitors. The devices may be provided with one or more NFAT inhibitors in addition to, or in replacement of, the bisphosphonate-hydroxyapatite combinations described herein. The present invention provides that such NFAT inhibitors are also effective to reduce or prevent osteolysis. Such NFAT inhibitors may comprise a protein inhibitor, a small molecule inhibitor, or combinations thereof. Non-limiting examples of NFAT inhibitors that may be used in the present invention include AKAP79, CABIN protein, CHP, MCIP1,2,3 proteins, cyclosporin A, FK506, and combinations thereof.

NFAT inhibitors are generally known to bind calcineurin and suppress dephosphorylating activity. There are at least four protein inhibitors that are known to prevent NFAT nuclear translocation, namely, AKAP79 (which is a scaffold protein that prevents calcineurin substrate interactions), CABIN protein (which blocks calcineurin activity), CHP (which is a calcineurin B homolog), and MCIP1,2,3 proteins (which have been shown to prevent NFAT2 phosphorylation and nuclear import). Two well-known NFAT small molecule inhibitors are cyclospprin A and FK506. The small molecule inhibitors, namely, cyclosporin A and FK506, have been shown to indirectly repress NFAT by inhibiting calcineurin activity.

NFAT inhibitors are further known to act as immunosuppressants by inhibiting alloreactive T-cells. NFAT inhibitors are often provided to patients in order to prevent graft rejection (and to provide reduced joint erosion and disease progression). More recently, 3,5-bistrifluoromethylpyrazole (BTP) derivatives have been shown to inhibit Th1 and Th2 cytokine gene expression. Thus, such molecules may further serve as NFAT inhibitors, and be provided to orthopaedic or orthodontic implantable devices in accordance with the present invention.

The following example is provided to further illustrate the methods, devices, and compositions of the present invention, as well as certain physical properties of the devices described herein. This example is illustrative only and is not intended to limit the scope of the invention in any way.

### EXAMPLES

### Example 1

### Determination of Optimal Zoledronate Concentration

A preliminary study was performed to determine an optimal concentration range of zoledronate for effectively reducing the pre-osteoclast population, while simultaneously having a less harmful effect on osteoblasts. Cell death assays were conducted using mouse osteoblasts and osteoclast precursor cells at different concentrations of zoledronate (Bedford Laboratories, OH).

MC3T3 cells, which are well-established mouse calvarial cells of osteoblastic lineage, and RAW 264.7 mouse macrophage-like cells, which are well-known to form osteoclasts, were purchased from the American Type Culture Collection and Tissue (Manassas, Va.). MC3T3 and RAW 264.7 cells were grown in Dulbecco's Modified Eagle Medium supplemented with 10% fetal bovine serum, 100 μ/mL penicillin, 100 mg/mL streptomycin, and 0.1% fungizone (amphotericin B). The cells were kept at 37° C. in a humidified atmosphere of 5% CO₂ and 95% air.

After treating the cells with zoledronate at concentrations of 0, 5, 10, 50, and 100 μM for 24 hours, the cells were pelleted by centrifugation and resuspended with 5 mL of PBS. Cell number was measured, and the cells were centrifuged and resuspended in binding buffer (A.G. Scientific, San Diego, Calif.) to a final cell density of 5×10⁵ cells/mL. 5 mL of annexin V-FITC (A.G. Scientific, San Diego, Calif.) was added to 195 mL of cell suspension and incubated for 10 minutes. The cells were centrifuged, washed with PBS and resuspended again in 190 mL of binding buffer. Ten mL of 20 mg/mL propidium iodide (A.G. Scientific, San Diego, Calif.) was added to the cell suspension, and flow cytometry was performed using a FACS Calibur (Becton Dickson, Franklin Lakes, N.J.). Monoparametric cytograms of annexin V-FITC fluorescence (FL1) versus number of events were created using the CellQuest program gating for living cells and excluding dead cells on the basis of their propidium iodide uptake.

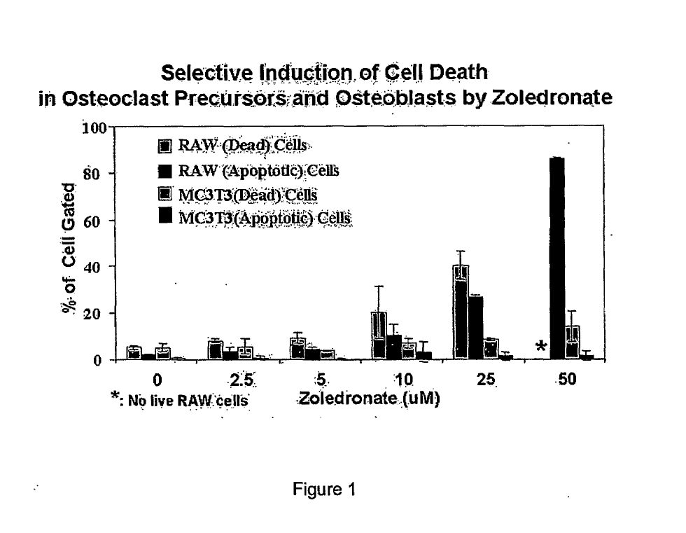

The foregoing experiment showed that using 50 μM of zoledronate induced 100% apoptosis in RAW cells (FIG. 1). Therefore, a 50 μM zoledronate solution was determined to be the optimal concentration. These data further suggest, however, that a range of 30-50 μM of zoledronate exhibits differential cytotoxic effects on osteoclast precursors and osteoblasts.

### Example 2

### First In Vivo Study

In this example, using a rodent osteolysis model, intramedullary femoral implants coated with effective amounts of zoledronate (a N-containing bisphosphonate) and hydroxyapatite were inserted into the femurs of rats. As shown below, the zoledronate- and hydroxyapatite-coated intramedullary implants were shown to reduce osteolysis adjacent to the implant site and to improve incorporation of the implant into the host bone.

More particularly, sterile ultrahigh molecular weight polyethylene (UHMWPE) wear debris particles (0.5 cc, 0.1-3 micron particle size) were introduced into the intramedullary cavity of bilateral femurs of 8 rats, which served as the control group in this example. The particles were introduced after reaming the femurs with an 18 gauge needle at the knee joint. Sterile wear debris particles were used in this example to stimulate a wear debris reaction and promote osteolysis. Hydroxyapatite-coated plastic pins and hydroxyapatite-coated titanium pins (1.5 mm×20 mm, Stryker, Kalamazoo, Mich.) were then inserted into the intramedullary canal of the right femur and left femur, respectively.

The test group, which consisted of 8 rates, also had 0.5 cc of sterile UHMWPE wear debris particles introduced into the femoral intramedullary canal after reaming with an 18 gauge needle at the knee joint. The hydroxyapatite-coated plastic pins and hydroxyapatite-coated titanium pins were presoaked in a zoledronate solution (80 mg/mL concentration; 30-50 μM of zoledronate) (Novartis Pharmaceuticals, East Hanover, N.J.) for 15 minutes prior to insertion into the right and left femurs, respectively.

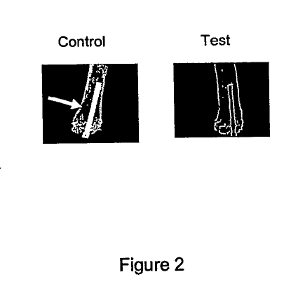

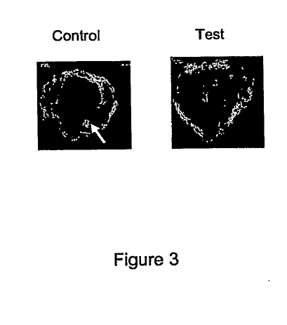

The rats of the test and control groups were sacrificed at 24 weeks following insertion of the pins and the femurs were retrieved. Faxitron radiographs (Faxitron, Wheeling, Ill.) of the femurs taken from the test and control rats were obtained. The Faxitron radiographs showed significant osteolysis around the titanium implant in the control rats, but not around the titanium implants in the test rats (FIG. 2). The femurs with plastic pin implants (the right femurs) were imaged by micro-CT (Stratec XCT-3000, Stratec Medizintechnik GmbH, Pforzheim, Germany). The micro-CT scans also showed significant osteolysis around the plastic implants in the control rats, but not around such implants in the test rats (FIG. 3).

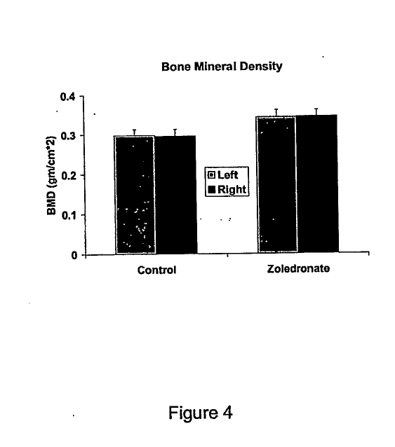

Osteolysis was quantitatively evaluated by measuring the overall bone mineral density of the femurs in the test and control rats, using dual-energy x-ray absorptiometry (DEXA) scans. This analysis demonstrated a statistically significant difference between the overall bone density of the femurs in the control and test rats (i.e., rats provided with zoledronate-treated pins, p<0.0001) (FIG. 4). No difference, however, was found between the left and right femurs within the test and control rats (i.e., plastic pin vs. titanium pin) (p=0.87) (FIG. 4).

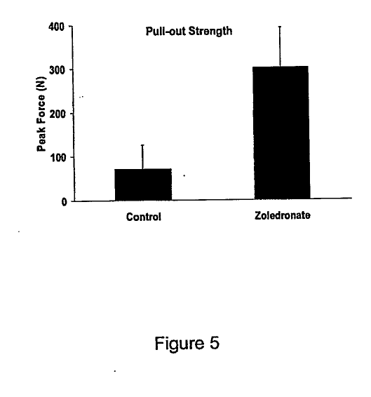

Incorporation of the implants by the host bone was evaluated by “pull-out strength testing” of the titanium pins (left femurs) (MTS 858 Bionix, MTS Systems Corporation, Minneapolis, Minn.). Such “pull-out strength testing” measures the amount of force (i.e., pull force) that must be applied to displace (or remove) the implant from the bone. Such biomechanical pull-out testing demonstrated that the zoledronate-treated group required a statistically higher (p<0.0001) peak force to extract the titanium pin implants (FIG. 5), as well as significantly higher (p=0.0085) pullout energy (5.71±3.42 N-m) compared to the control (1.61±1.65 N-m), indicating better incorporation.

Particle wear debris reactions are a significant component of osteolysis and aseptic implant loosening. The above in vivo example of a rodent femur model demonstrates that pretreatment of hydroxyapatite-coated intramedullary pins with zoledronate reduces the impact of wear debris reactions and leads to improved bone density and better incorporation of the implant into host bone. In addition, the topical application and localized delivery of such bisphosphonates to the target bone reduce the potential risks of systemic delivery of such compositions.

### Example 3

### Second In Vivo Study

Experimental Design and Surgical Procedure. In this example, a polyethylene particle-induced osteolysis rodent femur model was used to examine the advantages of using a HA-zoledronate composite on the implant surface for osseous integration and implant stability in the presence of UHMWPE particles. Two critical parameters were examined, namely, peri-implant bone quality (osteolysis) and osseointegration (implant stability).

All procedures involving animals were approved by the Institutional Animal Care and Use Committee in accordance with the Association for the Assessment and Accreditation of Laboratory Animal Care guidelines (Columbia University Animal Proctocol AC-AAM5306). In this example, a rodent femur intramedullary nailing model was used with UHMWPE wear debris particles instilled into the intramedullary canal after reaming the femur to stimulate a wear debris reaction. Thirty-two six-month-old adult male Sprague-Dawley rats (weight, 400-500 gm) were purchased (Charles Laboratory, Cambridge, Mass.).

The 32 rats were divided into two study groups, each composed of 16 rats. Sixteen randomly selected rats served as a control group, which received HA-coated nails. The remaining 16 rats received HA-coated nails with zoledronate and served as the experimental group. Eight rats in each group were sacrificed at 6 weeks following the intramedullary nailing of HA-coated implants and another 8 rats from each group were sacrificed 6 months following the surgery. Since zoledronate does not bind to metallic surfaces and HA coating changes the smoothness of the intramedullary implant, there was not an experimental group in which smooth, uncoated nails were tested.

After induction of general anesthesia using ketamine (60 mg/kg) and xylazine (5 mg/kg), hair over the knee was removed with a clipper. Right and left knee regions were prepared with 70% alcohol and 1% Betadine solution. A one centimeter longitudinal skin incision was made over the anterior aspect of the distal femur. A medial parapatellar approach was made to expose the intercondylar notch with the knee in flexion. An entry hole was made with a 18-gauge needle. The distal rat femora were reamed with an 18-gauge needle, and then Ceridust® UHMWPE particles (0.1 mL of 50 vol. %; 0.1 to 3 micron in size; Clariant GmbH, Germany) were introduced into the intradmedullary canal prior to implantation.

In the control group, a radiolucent UHMWPE intramedullary nail, which was coated with HA, was implanted into the right distal femur in a retrograde manner, while a HA-coated titanium intramedullary nail was implanted into the left distal femur. The joint capsule was repaired using a 2-0 Vicryl® suture (Johnson & Johnson, New Brunswick, N.J.). The skin was repaired with metal staples. Rats were allowed weight bearing as tolerated in their cages, while subcutaneous injections of buprenorphine (0.05 mg/kg) were given once a day for two post-operative days. Activity level, food intake, the condition of the surgical wound, and clinical signs of infection were monitored daily.

The purpose of using the UHMWPE nail was to minimize imaging artifacts inherent in metal implants for a more accurate analysis of dual energy x-ray absorptiometry (DXA) and microcomputed tomography (micro-CT) analyses, which are describe below. The metallic nail was used for biomechanical testing. The intramedullary nails, 1.4 mm in diameter and 20 mm in length, were custom-made for this study (Stryker, Mahwah, N.J.). For the test group, the aforementioned surgical procedures were performed using a HA-coated nail that was soaked in 50 μM zoledronate for 5 minutes at room temperature and then air dried.

All the femora were harvested, together with the implants, at 6 weeks and 6 months. The animals were sacrificed with the use of inhalant anesthesia, administered by placing the animals in a glass-covered tank with a sponge soaked with 1 mL of halothane, and an overdose of euthanasia solution (100 mg/kg of Beuthanasia-D, administered intraperitoneally). The retrieved left femora with titanium pins were wrapped in 0.15 M saline solution-soaked gauze, placed in labeled plastic bags, and stored at −25° C. until biomechanical testing was performed, as described below. The right femora with the UHMWPE pins were also fresh frozen in the same manner until undergoing micro-CT. Radiographs were obtained using a Faxitron machine (Faxitron X-Ray, Wheeling, Ill.; 40 kV for 30 seconds). The high-resolution radiographs were used to verify the intermedulary location of the implants in the left femur.

Dual Energy X-ray Absorptiometry (DXA). The right femora with UHMWPE pins and the left femora with titantium pins were imaged by DXA. The distal region of the bone extending from the distal end of the femur to the proximal end of the implant was selected for density analysis to focus on the area of the femur most immediate to the implant. The area of bone, bone mineral density, and bone mineral content were measured. A correction was made to the bone mineral content and bone mineral density of the left femora to account for the effect of the titanium pin. No correction was needed for the right femora because of the UHMWPE pin. A three-way ANOVA, with time (6 weeks versus 6 months) and treatment (control versus zoledronate) as non-repeated factors and leg as a repeated factor, was performed for the amount of bone area, bone mineral content, and bone mineral density.

The area of bone was found to be significantly greater (p<0.0029) for the 6 month group (1.284±0.067 mm²) than for the 6 week group (1.209±0.079 mm²). There was no difference (p=0.1222) between the bone areas in the control group (1.228±0.080 mm²) and the zoledronate group (1.265±0.081). There was also no difference (p=0.5898) in bone area between left (1.243±0.077 mm²) and right leg (1.250±0.088 mm²).

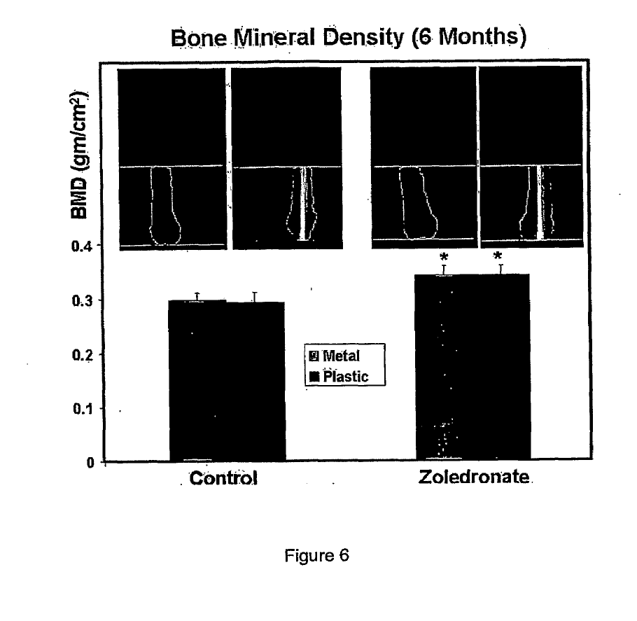

The bone mineral content of the 6 month group (0.462±0.061 g) was significantly greater (p<0.0001) than that of the 6 week group (0.338±0.057 g). The bone mineral content of the zoledronate group (0.417±0.064 g) was significantly (p<0.0001) greater than that of the control group (0.333±0.046 g). There was no difference in bone mineral content between left and right legs (p=0.2635). The bone mineral density was significantly greater (p<0.0001) for the 6 month group (0.320±0.040 g/cm²) than for the 6 week group (0.278±0.033 g/cm²). The bone mineral density was also significantly greater (p<0.0001) for the zoledronate group (0.328±0.036 g/cm²) than for the control group (0.271±0.025 g/cm²)(FIG. 6). There was no difference (p=0.1760) in bone mineral density between legs.

Micro-computed Tomography (micro-CT). The right femura with UHMWPE pins were imaged using a quantitative CT (Stratec XCT-3000), running the XCT Series software, version 5.21 (Stratec Medizintechnik GmbH, Pforzheim, Germany). Slice thicknesses were 1.0 mm. Voxel size was 0.1×0.1×1.0 mm. Three sequential axial images were obtained at 10 mm, 15 mm, and 20 mm from the distal end of the femur. The relative area of bone and bone mineral density for each of the images were obtained.

To focus on the site-specific effect of the zoledronate treatment, the images of the micro-CT slices were further analyzed using ImageJ, a public domain Java imaging-processing program inspired by NIH Image (http://rsb.info.nih.gov/ij/). An annulus was created and centered over the pin, with the 1.4 mm diameter of the pin as the inner diameter of the annulus and an outer diameter of 2 mm. The area of the annulus was thresholded at 290 mg/cm³ to differentiate bone from soft tissue and the area of bone within the annulus area was obtained for each of the slices.

A repeatability study was performed for this technique by repeating the procedure for all three slices on several randomly selected rats from both experimental groups and for both 6 week and 6 month groups. The estimated thresholded area was found to be repeatable to 0.030 mm² for the 6 week group and 0.025 mm² for the 6 month group, which were 3.5% and 1.7%, respectively, of the average mean thresholded areas of the rats used in the repeatability study.

A three-way ANOVA, with time (6 weeks versus 6 months) and treatment (control versus zoledronate) as non-repeated factors and image location (three levels) as a repeated factor, was performed for the amount of bone area. In addition, a linear correlation was performed between the amount of bone area in the annulus to pull-out strength and total energy to pull-out for both the 6 week groups and the 6 month groups.

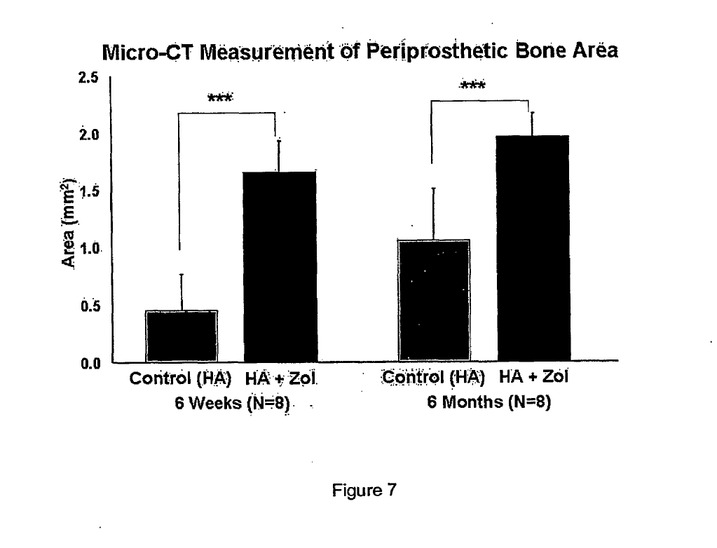

The amount of bone mass in the periprosthetic area was found to be significantly greater (p<0.0001) for the zoledronate group (2.388±0.960 mm²) than for the control group (0.933±0.571 mm²). The amount of bone mass was also found to be significantly greater (p<0.0001) for the 6 month group (1.517±0.571 mm²) than for the 6 week group (1.052±0.673 mm²). The effect of time was found to be more pronounced for the control group than for the zoledronate group (p=0.030), as can be seen in FIG. 7. There was no difference in periprosthetic bone mass between the three locations at which the images were taken (p=0.05).

Biomechanical Testing. The left femora of both the 6-week and 6-month control and treatment groups were subjected to biomechanical testing following the imaging studies described herein. The number of freeze-thaw cycles was kept consistent prior to mechanical testing. The bones were kept moist throughout each imaging study and during mechanical testing with 0.15 M NaCl. During storage and transit between imaging sites, the bones were wrapped with saline soaked gauze and kept at −78° C.

Each femur was potted in a ⅛″ galvanized steel threaded pipe nipple. A 1.4 mm hole was drilled through the pipe and bone and a 1.4 mm wire brad was passed through the pipe and bone below the level of the intramedullary pin. The pipe was filled with Rockite® expansion cement (Hartline Products Col, Inc., Cleveland, Ohio) to secure the bone to the pipe. The bone was kept moist throughout the potting process. Once potted, the distal end of the femur was carefully ground down until 3.5 mm of the intramedullary pin was exposed.

The potted bone was threaded into a testing jig, which allowed translation in the two orthogonal directions perpendicular to the long axis of the femur. The test jig included a ball joint which allowed internal-external, flexion-extension, and varsus-valgus rotations. The five degree-of-freedom set-up ensured that the direction of pull was in-line with the axis of the intrameduallary rod. The test jig was mounted in a 858 Bionix® servohydraulic material tester (MTS, Eden Prairie, Minn.), and the exposed pin gripped by a precision drill chuck attached to a 5 kN load cell to ensure that the intramedullary pin was centered and the direction of pull was in-line with the axis of the pin. The intramedullary pin was pulled out at a constant rate of 2 mm/min. The displacement was provided by the stroke linear variable differential transducer (LVDT) of the MTS and the load by the MTS load cell set at its 500 N range, both as a function of time. Energy to complete pullout was obtained by a trapezoidal integration of the force-displacement curve.

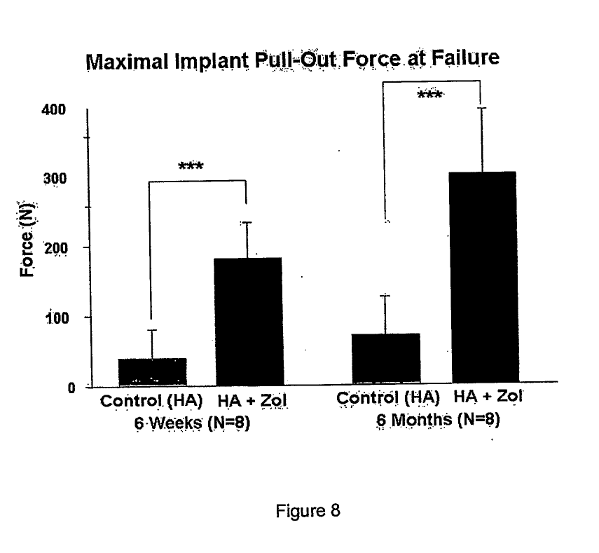

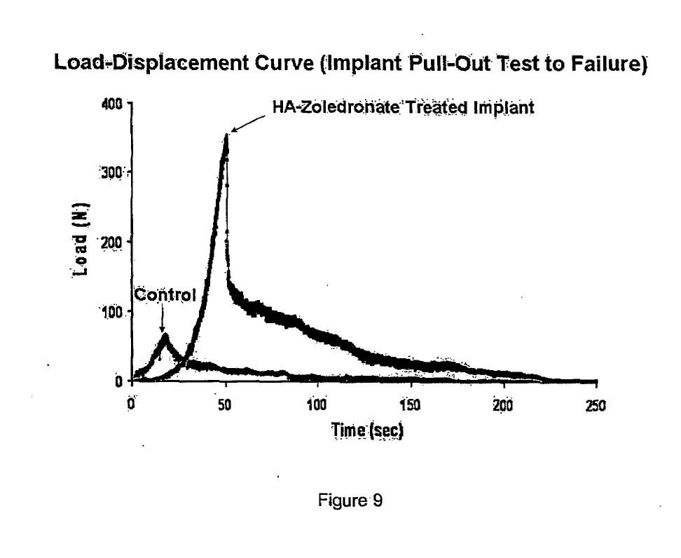

The average peak pullout force of the treated femora (241.0±95.1 N) was significantly greater (p<0.0001) than that of the controls (55.6±49.0 N)(FIG. 8). See also FIG. 9. The 6-month femora exhibited a statistically greater (p=0.0022) average peak force (185.8±139.5 N) than was observed for the 6-week femora (110.8±85.5 N)(FIG. 8). There was no statistical difference in the effect of treatment between the 6 week and 6 month groups (p=0.0553).

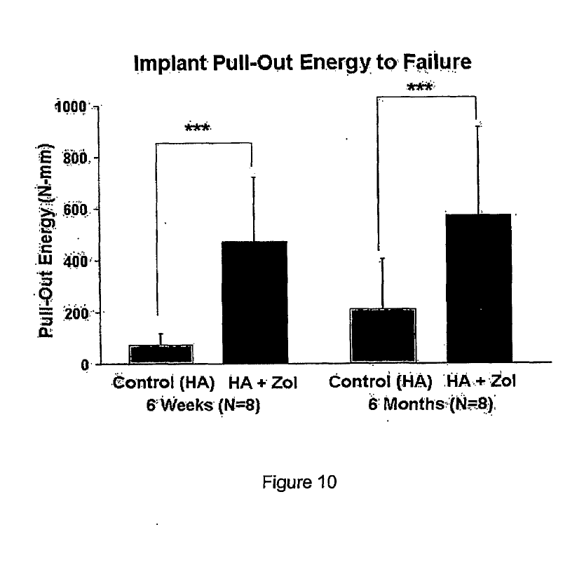

The energy required to completely pull the pin out of the bone was significantly greater (p<0.0001) for the treated femora (521.6±293.8 N-mm) than for the controls (142.2±152.1 N-mm)(FIG. 10). No statistically significant difference (p=0.1616) was found between the total energy to pull-out between the 6-weeks (272.6±269.1 N-mm) group and the 6-months group (391.2±326.1 N-mm). The effect of treatment was slightly greater for the 6-week group (a 348.7% increase) than for the 6-month group (a 342.3% increase). However, this was not statistically significant (p=0.0553). A similar trend was observed for the total energy to pull-out, with a 542.8% increase for the 6-weeks group as compared to a 170.7% increase for the 6-month group. Though the difference between groups was large, this trend was also not statistically significant (p=0.8198) because of the large variance observed in this measurement.

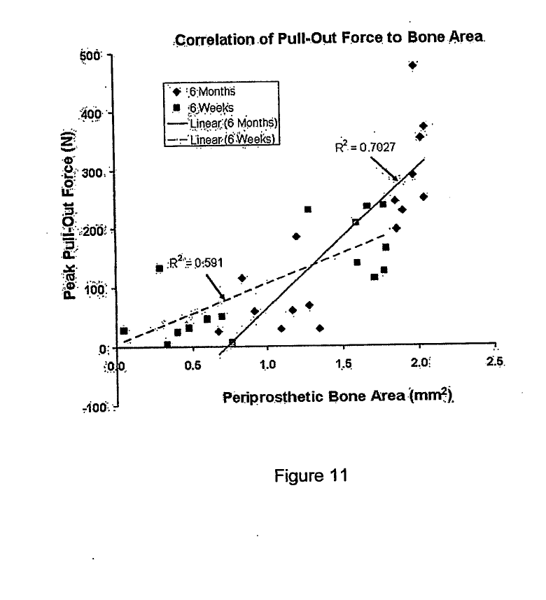

Bone Mass versus Mechanical Strength. The Pearson correlation coefficient between the bone area in the annulus and the peak pull-out force was 0.769 (p=0.0005) for the 6-week group and 0.838 (p<0.0001) for the 6-month group. The slope of the 6-month group (238.1 N/mm²) was found to be statistically greater (p=0.0336) than the slope of the 6-week group (101.1 N/mm²) as shown in FIG. 11. The Pearson correlation coefficient between the bone area in the annulus and the total energy for pull-out was 0.692 (p=0.0030) for the 6-week group and 0.595 (p=0.0152) for the 6-month group. The slopes of the 6-month group (394.6 N/mm) and the 6-week group (286.4 N/mm) were not statistically different (p=0.9326).

As depicted in this example, both bone density measurements and the biomechanical study demonstrate that presoaking HA-coated implants in zoledronate solution showed significantly higher bone mass and superior pin pullout strength. Further, the ease with which zoledronate can be applied onto HA-coated implants simply by soaking it makes the findings from the above study readily applicable in clinical settings. The benefits of adding zoledronate were also shown to be long lasting. Even after six months, the periprosthetic bone density was significantly higher in the treatment group with correspondingly higher mechanical strength as measured by pin pullout force. The long-term persistence of this effect is promising when considered in the context of the clinical setting, such as primary arthroplasties, revision surgeries, osteoporosis and metastatic bone cancers.

Our pull-out test showed zolendronate improves implant fixation. The foregoing model is clinically relevant to examine whether bisphosphonate can reduce implant loosening by preventing osteolysis, a major cause of the loss of osseointegration. Indeed, the HA-zoledronate composite group required significantly higher pull-out energy and higher peak pull-out force when compared to the non-treated group. Since all of the pins were the same diameter and the same amount of the pin extended from the bone for all the femurs during the pull-out tests, the average peak shear stress is directly proportional to the average peak pullout force as a result of the interaction between the host bone and HA-coated implants.

In comparing the peak pull-out force between the six-week versus the six-month group, the foregoing shows that the six-month group required significantly higher force than the six-week group. In other previous studies, however, since osseointegration is completed by 6 weeks, no significant differences in terms of pull-out force after 6 weeks were observed. The control groups of the current study did not show any significant difference of pull-out force at different time points, which is consistent with the results of previous studies. Interestingly, zolendronate improved the osseointegration gradually until 6 months, which demonstrates that even a simple soaking of zolendronate affects osseointegration in a long-term manner.

### Example 4

### NFATc1 Inhibitors Prevent Osteolysis

The cause of osteolysis has been studied for many years, but has remained relatively unclear. The lack of clarity regarding the cause(s) of osteolysis may exist for several reasons. For example, it is difficult to assess the effects of wear particles in bone tissue where the disparate cells respond diversely against particles. Thus, there has been a continuing need to elucidate a major molecular signaling pathway induced by wear particles that determines the synergistic cooperation among osteoblasts, macrophages, and osteoclasts and leads to osteolysis.

The following example will focus on osteoblasts and macrophages and the roles thereof in osteolysis. Osteoblats and macrophages were the focus of this example because they have been known as major sources for the initiation of both RAW 264.7 cells and MC3T3-E1 cells. More specifically, in this example, the relationship between one of the downstream regulators of calcium signaling, calcineurin/NFAT, and the cytokine expression in both RAW 264.7 and MC3T3-E1 cells was verified.

Cell culture. MC3T3-E1 cells were purchased from the American Type and Culture Collection (ATCC, Manassas, Md.). RAW cells were cultured in Dulbecco's Modified Eagle Medium (DMEM) with 10% fetal bovine serum (FBS).

Particle preparation and analysis. Ultra-high molecular weight polyethylene (UHMWPE) particles were provided by Stryker Corporation from serum-contained hip simulators (1 Hz, 106 cycles). Affatato's method was followed in isolating the particles. Endotoxin levels were checked by Limulus assay (E-Toxate kit, Sigma-Aldrich Chemical Co., St. Louis, Mo.).

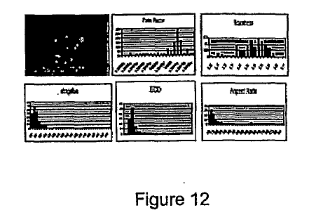

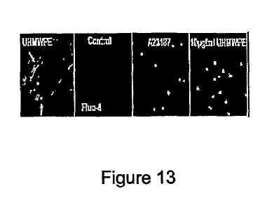

Intracellular calcium imaging study. MC3T3-E1 cells were incubated in 2 mM fluo-4/AM media for 30 minutes at 37° C., 5% CO₂. The UHMWPE wear particles were analyzed by NIH Image J software. As shown in FIG. 12, the shapes and sizes of such particles were similar to wear particles from pseudomembranes. Referring to FIG. 13, the MC3T3-E1 cells were observed to be engulfed in clinically relevant amounts of the UHMWPE wear particles. As shown therein, the UHMWPE elevated intracellular calcium levels in the MC3T3-E1 cells.

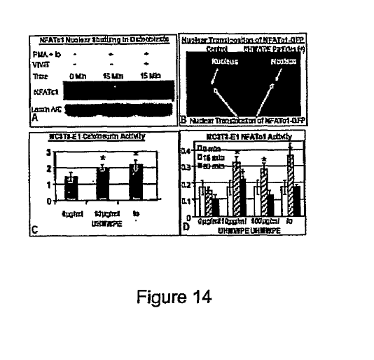

Determination of calcineurin/NFAT activation. Calcineurin activity was measured with a calcineurin activity assay kit (Calbiochem, Calif.). Cells were transfected with NFATc1 and NFATc1-EGFP cDNA constructs using Fugene 6 (Roche, Ind.). Referring to FIG. 14(A), the stimulation of the calcium/calcineurin/NFAT axis resulted in an increased nuclear fraction of NFATc1. As shown in FIG. 14(A), the VIVIT peptide—an inhibitor of NFAT—abolished NFATc1 activation. Referring to FIG. 14(B)-(D), UHMWPE particles were shown to increase the activity of calcineurin (p<0.05), resulting in the nuclear translocation of NFATc1 and increased NFATc1 activity (p<0.05) in MC3T3-E1 cells.

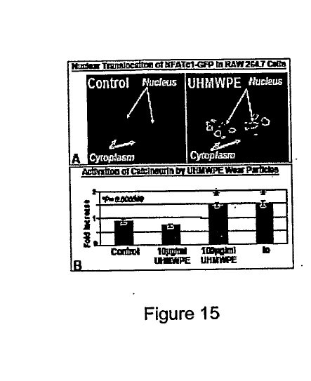

Nuclear translocation of NFATc1-GFP was also measured in RAW 264.7 cells. First, NFATc1-GFP cDNA was transfected into RAW 264.7 cells. Next, nuclear and cytoplasmic fractions were obtained using a nuclear extraction kit (Active Motif, CA). Referring to FIG. 15(A), the nucleus does not contain the green fluorescent protein (GFP) and is black at its resting stage. 10-15 minutes after the addition of wear particles, however, GFP-NFATc1 proteins were translocated into the nucleus. Referring to FIG. 15(B), the wear particles were shown to activate calcineurin in the RAW 264.7 cells.

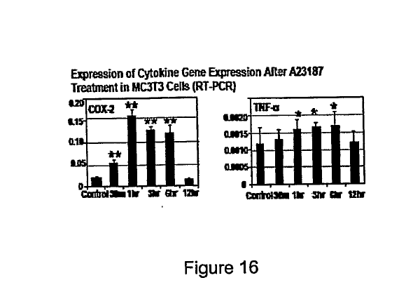

Wear particles and calcium signaling induce cytokine expression in macrophages and osteoblasts. Cytokine gene expression was measured following A23187 treatment in MC3T3 cells by Quantitative-RT PCR. RNA was isolated from the cells using an RNeasy Mini Kit (Qiagen, Valencia, Calif.), and Quantitative-RT PCR was performed using a SYBR Green master mix (Roche, Ind.). Referring to FIG. 16, the calcium signaling pathway was found to increase COX-2 and TNF-alpha expression 1 to 6 hours after stimulation in the MC3T3 cells (relative to the controls)(N=3; mean+1-standard deviation).

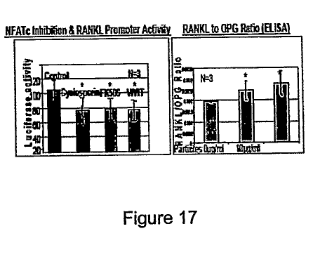

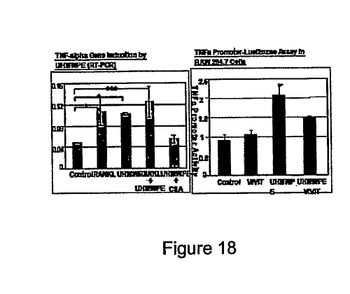

The calcineurin/NFATc1 axis was then inhibited by providing the cell culture with 1 μM of an NFAT inhibitor, e.g., Cyclosporine A. Referring to FIG. 17, inhibiting the calcineurin/NFATc1 axis was shown to decrease RANKL promoter activity (*p<0.05), whereas the wear particles were shown to increase RANKL production and the RANKL/OPG ratio in the MC3T3 cells (*p<0.05). Referring to FIG. 18, wear particles were shown to increase TNF-alpha expression in RAW 264.7 cells, whereas cyclosporine A prevented particle-induced TNF-alpha gene expression (N=3; *p=0.03; **p=0.05; ***p=0.04). TNF-alpha gene promoter activity was also shown to increase 3 hours after the addition of the particles. VIVIT (at 1 μM) was shown to significantly block TNF-alpha gene promoter activity (FIG. 18).

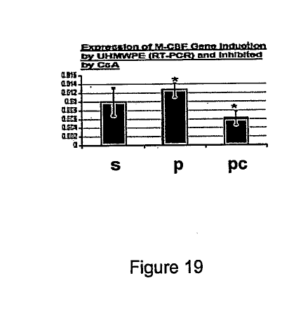

Finally, M-CSF gene induction by UHMWPE was measured, as well as the effects of cyclosporine A on such induction. Referring to FIG. 19, M-CSF expression increased one day after the application of the wear particles. The addition of cyclosporine A, however, appeared to block M-CSF expression (N=3; *p<0.01).

As shown in the above Example 4, clinically relevant amounts of UHMWPE particles elevate intracellular calcium and clacineurin activity in MC3T3-E1 and RAW cells. Such activity was shown to enhance NFATc1 nuclear translocation in both types of cells. In light of the foregoing data, the calcium/calcineurin/NFATc1 axis seems to regulate cytokine expression in both types of cells. Although NFATc1 regulated different cytokines in MC3T3-E1 and RAW cells, most of them, such as COX2, TNF-alpha, M-CSF, and RANKL, are well-known to play major roles in osteolysis. Accordingly, the foregoing demonstrates the utility of NFATc1 inhibitors in preventing osteolysis.

Although illustrative embodiments of the present invention have been described herein, it should be understood that the invention is not limited to those described, and that various other changes or modifications may be made by one skilled in the art without departing from the scope or spirit of the invention.

1. A method of improving the incorporation of an implantable device into a bone of a host in need thereof, which method comprises implanting the device into the bone of a host, wherein the device is at least partially made of a non-metallic material and disposed on at least one surface of the device is an amount of hydroxyapatite and bisphosphonate, which in combination, are effective to reduce osteolysis and improve incorporation of the implant into the host bone compared to an implant without the hydroxyapatite and/or bisphosphonate.

2. The method of claim 1, wherein the implant or a component thereof is made of a polymeric material.

3. The method of claim 2, wherein the polymeric material is polyethylene.

4. The method of claim 3, wherein the bisphosphonate is a nitrogen-containing bisphosphonate.

5. The method of claim 4, wherein the nitrogen-containing bisphosphonate is selected from the group consisting of pamidronate, neridronate, olpadronate, alendronate, ibandronate, risedronate, zoledronate, and mixtures thereof.

6. The method of claim 1, wherein the device is an orthopaedic or orthodontic implantable device.

7. The method of claim 1 further comprising an amount of an NFAT inhibitor effective to reduce or prevent osteolysis, which inhibitor is disposed on at least one surface of the device.

8. The method of claim 7, wherein the NFAT inhibitor is a protein inhibitor or a small molecule inhibitor.

9. The method of claim 7, wherein the NFAT inhibitor is selected from the group consisting of AKAP79, CABIN protein, CHP, MCIP1,2,3 proteins, cyclosporin A, FK506, and combinations thereof.

10. A method of making a device for implanting into a bone of a host in need thereof, which method comprises contacting, prior to implantation, the device or at least one surface thereof with an amount of bisphosphonate and hydroxyapatite, which, in combination, are effective to reduce osteolysis adjacent to the implant site and to improve incorporation of the implant into the host bone compared to a device without the hydroxyapatite and/or bisphosphonate.

11. The method of claim 10, wherein the contacting step comprises immersing the device or a surface thereof in a composition comprising a mixture of hydroxyapatite and bisphosphonate.

12. The method of claim 10, wherein the contacting step comprises immersing the device or a surface thereof first in a composition comprising hydroxyapatite, allowing the hydroxyapatite to dry, and then immersing the hydroxyapatite-coated device in a composition comprising bisphosphonate.

13. The method of claim 10, wherein the bisphosphonate is selected from the group consisting of pamidronate, neridronate, olpadronate, alendronate, ibandronate, risedronate, zoledronate, and mixtures thereof.

14. The method of claim 10, further comprising contacting the device or at least a surface thereof with an amount of a NFAT inhibitor that is effective to reduce or prevent osteolysis.

15. The method of claim 11, wherein the composition further comprises an amount of a NFAT inhibitor that is effective to reduce or prevent osteolysis.

16. The method of claim 14, wherein the NFAT inhibitor is a protein inhibitor or a small molecule inhibitor.

17. The method of claim 16, wherein the NFAT inhibitor is selected from the group consisting of AKAP79, CABIN protein, CHP, MCIP1,2,3 proteins, cyclosporin A, FK506, and combinations thereof.

18. An orthopaedic or orthodontic implant, which comprises a surface or component that is at least partially made of a non-metallic material, wherein the surface or component is provided with an amount of hydroxyapatite and bisphosphonate effective to reduce osteolysis adjacent to a site where the implant is implanted and to improve incorporation of the implant into a host bone compared to an implant without the hydroxyapatite and/or bisphosphonate.

19. The orthopaedic or orthodontic implant of claim 18, wherein the surface or component of the implant is made of a polymeric material.

20. The orthopaedic or orthodontic implant of claim 19, wherein the polymeric material is polyethylene.

21. The orthopaedic or orthodontic implant of claim 20, wherein the bisphosphonate is a nitrogen-containing bisphosphonate.

22. The orthopaedic or orthodontic implant of claim 21, wherein the nitrogen-containing bisphosphonate is selected from the group consisting of pamidronate, neridronate, olpadronate, alendronate, ibandronate, risedronate, zoledronate, and mixtures thereof.

23. The orthopaedic or orthodontic implant of claim 18, wherein a surface or component of the implant is further provided with an amount of a NFAT inhibitor that is effective to reduce or prevent osteolysis.

24. The method of claim 23, wherein the NFAT inhibitor is a protein inhibitor or a small molecule inhibitor.

25. The method of claim 23, wherein the NFAT inhibitor is selected from the group consisting of AKAP79, CABIN protein, CHP, MCIP1,2,3 proteins, cyclosporin A, FK506 and combinations thereof.

26. A method of improving the incorporation of an implantable device into a bone of a host in need thereof, which method comprises implanting the device into the bone of a host, wherein the device is at least partially made of a non-metallic material and disposed on at least one surface of the device is an amount of a NFAT inhibitor, which is effective to reduce osteolysis and/or modulate cytokine expression compared to an implant without the NFAT inhibitor.

27. The method of claim 26, wherein the implant or a component thereof is made of a polymeric material.

28. The method of claim 27, wherein the polymeric material is polyethylene.

29. The method of claim 26, wherein the device is an orthopaedic or orthodontic implantable device.

30. The method of claim 26, wherein the NFAT inhibitor is a protein inhibitor or a small molecule inhibitor.

31. The method of claim 26, wherein the NFAT inhibitor is selected from the group consisting of AKAP79, CABIN protein, CHP, MCIP1,2,3 proteins, cyclosporin A, FK506 and combinations thereof.

32. A method of making a device for implanting into a bone of a host in need thereof, which method comprises contacting, prior to implantation, the device or at least one surface thereof with an amount of an amount of a NFAT inhibitor, which is effective to reduce osteolysis and/or modulate cytokine expression compared to an implant without the NFAT inhibitor.

33. The method according to claim 32, wherein the contacting step comprises immersing the device or a surface thereof in a composition comprising the NFAT inhibitor.

34. The method of claim 32, wherein the NFAT inhibitor is a protein inhibitor or a small molecule inhibitor.

35. The method of claim 32, wherein the NFAT inhibitor is selected from the group consisting of AKAP79, CABIN protein, CHP, MCIP1,2,3 proteins, cyclosporin A, FK506, and combinations thereof.

36. An orthopaedic or orthodontic implant, which comprises a surface or component that is at least partially made of a non-metallic material, wherein the surface or component is provided with an amount of a NFAT inhibitor, which is effective to reduce osteolysis and/or modulate cytokine expression compared to an implant without the NFAT inhibitor.

37. The orthopaedic or orthodontic implant of claim 36, wherein a surface or component of the implant is made of a polymeric material.

38. The orthopaedic or orthodontic implant of claim 37, wherein the polymeric material is polyethylene.

39. The method of claim 36, wherein the NFAT inhibitor is a protein inhibitor or a small molecule inhibitor.

40. The method of claim 37, wherein the NFAT inhibitor is selected from the group consisting of AKAP79, CABIN protein, CHP, MCIP1,2,3 proteins, cyclosporin A, FK506, and combinations thereof.

41. The method of claim 15, wherein the NFAT inhibitor is a protein inhibitor or a small molecule inhibitor.
